(19) United States
(12) Patent Application Publication
(10) Pub. No.: US 2025/0304898 A1
(43) Pub. Date: Oct. 2, 2025

(54) MULTI-STATION BIOREACTOR

(71) Applicant: PEPTREE INC., Gunpo-si (KR)

(72) Inventors: Sang Seok LEE, Seoul (KR); Jung Hoon AHN, Busan (KR)

(73) Assignee: PEPTREE INC., Gunpo-si (KR)

(21) Appl. No.: 19/018,743

(22) Filed: Jan. 13, 2025

(30) Foreign Application Priority Data
Apr. 2, 2024 (KR) 10-2024-0044935

(51) Int. Cl. C12M 3/00; C12M 1/34; C12M 1/36; C12M 3/06

(52) U.S. Cl. CPC C12M 23/44; C12M 27/16; C12M 41/12; C12M 41/34; C12M 41/36; C12M 41/48

(57) ABSTRACT

A multi-station bioreactor is proposed. The multi-station bioreactor includes a plurality of stations (10) on which a culture container (1) containing a culture solution is placed; and a driving part (30) generating a mechanical driving force, wherein the plurality of stations (10) is arranged to be vertically spaced apart from each other, and the driving force of the driving part (30) is transmitted to the plurality of stations (10) so that the plurality of stations (10) is moved at the same time.

### CROSS REFERENCE TO RELATED APPLICATION

The present application claims priority to Korean Patent Application No. 10-2024-0044935, filed Apr. 2, 2024, the entire contents of which are incorporated herein for all purposes by this reference.

### BACKGROUND OF THE INVENTION

### Field of the Invention

The present disclosure relates to a bioreactor having multiple layers of stations, which is advantageous for mass production.

### Description of the Related Art

Generally, bioreactors are used as the standard for culturing E. coli, and an appropriate environment for a culture bag is created with a plurality of tubes and sensors connected to the culture bag. Since E. coli cultivation is used as the standard for E. coli cultivation, there are many large-capacity bioreactors for E. coli cultivation. However, currently, bioreactors that use culture bags only exist as single culture bag products.

However, for pseudomonas, there are not many places where pseudomonas strains are available, and there are not many cases of commercially using pseudomonas. Therefore, there are no suitable bioreactors for pseudomonas. Pseudomonas and the like may be cultured by simply shaking the pseudomonas, and a bioreactor suitable for pseudomonas is also needed.

### SUMMARY OF THE INVENTION

An objective of the present disclosure is to provide a bioreactor suitable for mass production of microorganism.

According to an aspect of the present disclosure, there is provided a multi-station bioreactor including: a plurality of stations on which a culture container containing culture solution is placed; and a driving part generating a mechanical driving force, wherein the plurality of stations may be arranged to be vertically spaced apart from each other, and the driving force of the driving part may be transmitted to the plurality of stations so that the plurality of stations may be moved at the same time.

For the multi-station bioreactor described above, each of the plurality of stations may be rotatably hinged with a standing frame at facing two left and right hinge points of each station, the multi-station bioreactor further may include: a connecting rod connected at one end to the driving part, and connected to a connection point of each of the plurality of stations, the connection point being spaced apart from each of the two hinge points, and transmitting the driving force of the driving part to each of the stations.

For the multi-station bioreactor described above, each of the stations may be moved like a seesaw as the connection point may be rotated at a predetermined angle on each of the two hinge points by the connecting rod.

For the multi-station bioreactor described above, the driving part may include: a motor driven by electric energy; a reducer reducing and outputting rotation of the motor; and a rotating wheel coupled to an output terminal of the reducer, wherein one end of the connecting rod may be connected to circumference of the rotating wheel so that the end of the connecting rod may be circularly moved.

For the multi-station bioreactor described above, the driving part may include a vibrator generating vibration, and one end of the connecting rod may be coupled to the vibrator, and the connecting rod may transmit the vibration to the connection point.

For the multi-station bioreactor described above, the multi-station bioreactor may include a heating part provided on an upper surface of each station or a bottom of the culture container and consisting of a heating wire or a heating plate to heat the culture solution.

For the multi-station bioreactor described above, the multi-station bioreactor may culture pseudomonas.

For the multi-station bioreactor described above, the multi-station bioreactor may further include: a sensor sensing at least color of the culture solution in the culture container; and a control unit comprising a machine learning model that determines a cultivation level from sensing data of the sensor.

For the multi-station bioreactor described above, the machine learning model may be pre-trained with a data set including a data pair consisting of the sensing data of the sensor and the cultivation level.

For the multi-station bioreactor described above, the cultivation level may include an appropriate level, and when the machine learning model determines the cultivation level as the appropriate level, the machine learning model may stop the driving part or notify a user.

For the multi-station bioreactor described above, the cultivation level may include a lack of oxygen, and when the machine learning model determines the cultivation level as the lack of f oxygen, the control unit may increase the operation speed of the driving part or notify a user.

According to the present disclosure, the multi-station bioreactor has the advantage of culturing a large number of microorganisms such as pseudomonas with the simple and low-cost structure.

The multi-station bioreactor of the present disclosure can perform large-capacity cultivation without a rotating blade and the like, so the multi-station bioreactor has advantages such as no cell stress and damage, low cross-contamination possibility, and high process flexibility.

According to the present disclosure, the multi-station bioreactor has the advantage of facilitating up scaling.

According to the present disclosure, the multi-station bioreactor can easily respond to a lack of oxygen by using a sensor and machine learning, can automatically stop the cultivation in response to the completion of cultivation, and can notify the user.

### BRIEF DESCRIPTION OF THE DRAWINGS

FIG. 1 is a concept view (side view) illustrating an outline of a multi-station bioreactor according to the present disclosure.

FIG. 2 is a concept view (side view) illustrating an outline of a state in which the multi-station bioreactor according to the present disclosure is moved like a seesaw.

FIG. 3 is a perspective view illustrating a structure of the multi-station bioreactor according to an embodiment of the present disclosure.

FIG. 4 is a perspective view illustrating a driving part of the multi-station bioreactor according to the embodiment of the present disclosure.

FIG. 5 is a concept view (side view) illustrating a multi-station bioreactor according to another embodiment of the present disclosure.

FIG. 6 is a concept view (front view) illustrating a multi-station bioreactor according to another embodiment of the present disclosure.

### DETAILED DESCRIPTION OF THE DISCLOSURE

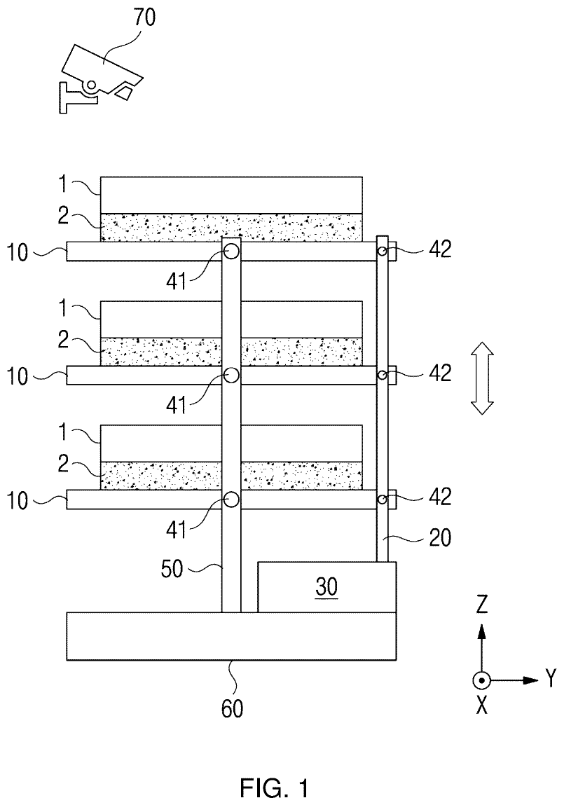

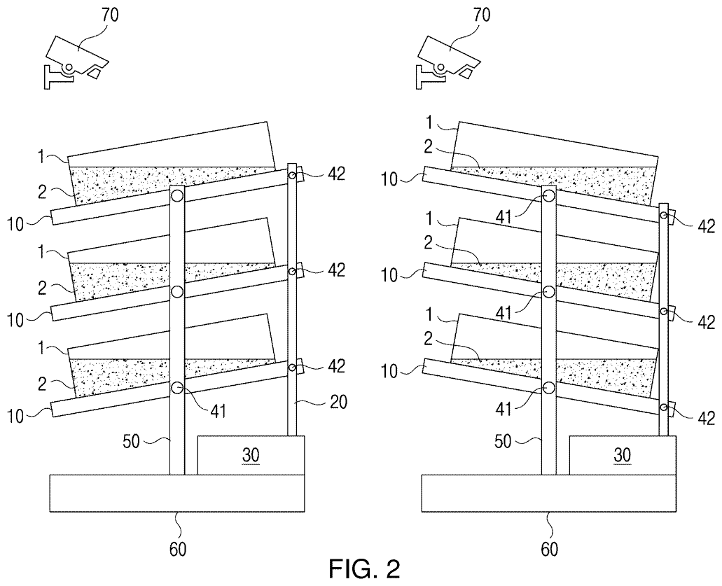

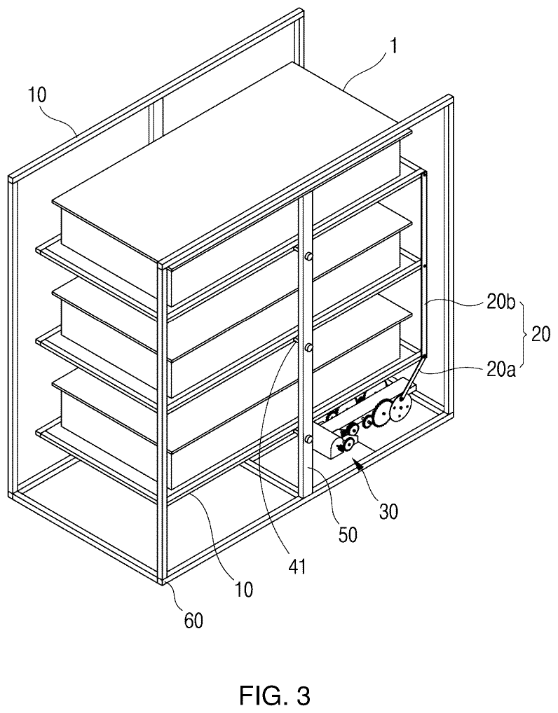

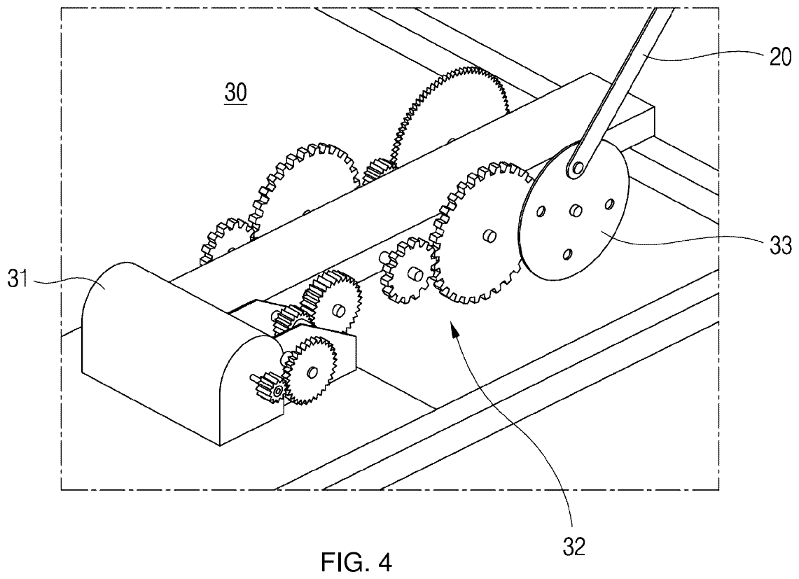

FIG. 1 is a concept view (side view) illustrating an outline of a multi-station bioreactor according to the present disclosure. FIG. 2 is a concept view (side view) illustrating an outline of a state in which the multi-station bioreactor according to the present disclosure is moved like a seesaw. FIG. 3 is a perspective view illustrating a structure of the multi-station bioreactor according to an embodiment of the present disclosure. FIG. 4 is a perspective view illustrating a driving part of the multi-station bioreactor according to the embodiment of the present disclosure.

According to the embodiment of the present disclosure, the multi-station bioreactor includes a plurality of stations 10, a connecting rod 20, a driving part 30, a plurality of hinges 41, a plurality of rotating connection parts 42, a frame 50, a base 60, and a sensor 70.

The plurality of stations 10 is vertically arranged at intervals, and each of the stations 10 may be provided for a culture container 1 containing the culture solution therein to be placed thereon. The culture container 1 may be a culture bag, a culture tank, etc., and may be provided to contain and maintain a culture solution 2 cultured. The culture container 1 may include a filter at an upper portion thereof to communicate with the outside space. A sensor that can sense dissolved oxygen levels of the culture solution 2 may be provided at the culture container 1.

Each of the stations 10 has a shape of a rectangular plate, a quadrangle frame, etc., that is for the culture container 1 to be placed thereon and may have a guide along an edge to prevent outward separation of the culture container 1. The frame 50 is a rod standing based on the base 60 at the bottom, and at least two left and right frames are vertically installed at left and right portions of each station 10. The frame 50 is a vertical rod that bears and supports the weight of the culture container 1 despite the movement of each station 10.

Each of the stations 10 is fixed to the vertically installed frame 50 by using two hinges 41 at the facing two left and right hinge points of each station 10 and hinged to be relatively rotatable to the frame 50. The hinges 41 are located at both edges in a transverse direction (X direction perpendicular to the drawing) of each station 10 for each station 10, thereby allowing each station 10 to be rotatable thereon.

Each of the stations 10 is rotatable on the two hinge points, and each station 10 is moved like the movement of a seesaw by the connecting rod 20 driven by the driving part 30.

One end of the connecting rod 20 is connected to the driving part 30, and the connecting rod 20 is connected to each station 10 at each rotating connection part 42 provided in each station 10 and rotatably connected thereto. At connection points spaced apart from the two hinge points of each station 10, each station 10 and the connecting rod 20 are connected to each other by each rotating connection part 42. Accordingly, the driving force of the driving part 30 is transmitted to each station 10.

In FIG. 1, the connecting rod 20 has a straight rod shape but may have different shapes. As shown in FIG. 3, the connecting rod may be a plurality of rods including a first connecting rod 20a connecting the driving part 30 to a first station 10 and a second connecting rod 20b connecting the second station 10 and remaining stations 10 to each other.

The driving part 30 generates a physical and mechanical driving force, and the driving force of the driving part 30 is transmitted to the plurality of stations 10 to move the plurality of stations 10 at the same time.

The driving part 30 according to the first embodiment includes a motor 31 driven by electric energy (referring to FIG. 4), a reducer 32 reducing and outputting speed of the motor, a rotating wheel 33 coupled to an output terminal of the reducer 32 and rotated. An end of the connecting rod 20 is connected to the circumferential surface of the rotating wheel 33 and circularly moved in response to the rotation of the rotating wheel 33.

The end of the connecting rod 20 is connected to the rotating wheel 33, and the connecting rod 20 is connected to each of the stations 10 at each connection point provided in a middle portion of the connecting rod 20. As the end of the connecting rod 20 is circularly moved along the circumference of the rotating wheel 33, the connection point (rotating connection part 42) is movable along a circular arc centered on each hinge 41, and each station 10 is moved like a seesaw. Each station 10 is rotated within a limited predetermined angle at the connection point to be moved like a seesaw.

The driving part 30 according to a second embodiment includes a vibrator generating vibrations. An end of the connecting rod 20 is coupled to the vibrator, and the connecting rod 20 transmits vibrations to each connection point. In the driving part 30 according to the second embodiment, the movement of each station 10 is smaller than the driving part 30 according to the first embodiment but has the advantage of not requiring the reducer and the like.

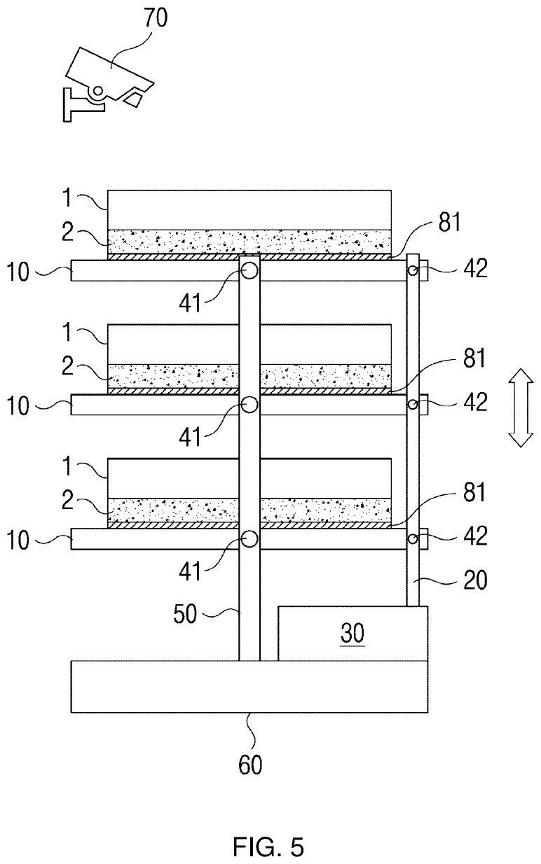

FIG. 5 is a concept view (side view) illustrating a multi-station bioreactor according to another embodiment of the present disclosure.

According to another embodiment of the present disclosure, the multi-station bioreactor includes a heating part 81 compared to the multi-station bioreactor described with reference to FIGS. 1 to 4. The heating part 81 is formed on the upper surface of each station 10 or on the bottom of the culture container 1 and consists of a heating wire or a heating plate to heat the culture solution in the culture container 1. Heating and heating temperature of the heating part 81 may be controlled by a control unit.

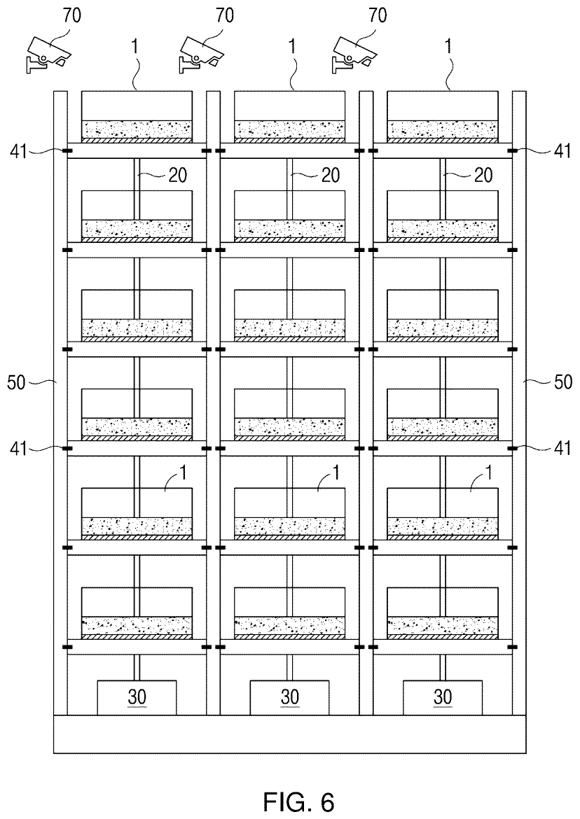

FIG. 6 is a concept view (front view) illustrating a multi-station bioreactor according to another embodiment of the present disclosure.

The multi-station bioreactor according to another embodiment of the present disclosure as illustrated in FIGS. to 5 includes a two-dimensional array in which a plurality of stations is arranged to be stacked vertically and several vertically arranged stations (hereinbelow, which will be called ‘station columns’) are arranged transversely. Two stations adjacent to each other may be provided while sharing a single frame 50. Stations included in each station column are driven by the single driving part 30 at the same time, and a number of driving parts 30 corresponding to the number of station columns should be provided.

According to the present disclosure, there is an advantage of being able to place the plurality of stations and the plurality of culture containers closely together in a narrow space, and cultivation start time and cultivation level can be set separately for each station column.

The multi-station bioreactor according to the present disclosure is optimized for culturing pseudomonas. Pseudomonas is used as a raw material for peptide cosmetics and health-functional foods or to adsorb mercury compounds in wastewater and reduce them into metallic mercury.

The control unit (not illustrated) controls the driving speed of the driving part 30 to control the vertical movement speed of the connecting rod 20, and each station is operated like a seesaw. Then, the culture solution 2 in the culture container 1 is moved transversely and a flow like waves occurs in the culture solution so that the culture solution is brought into contact with air and dissolved oxygen is introduced into the culture solution.

The sensor 70 senses the color of the culture solution in the culture container 1, and is a color sensor sensing the color of received light or a camera capturing an object and obtaining color pictures or color images. The control unit (not illustrated) includes a machine learning model receiving sensing data of the sensor 70 and determining a cultivation level from the sensing data. The machine learning model is pre-trained using a training data set. The machine learning model is pre-trained with a data set including a data pair consisting of the sensing data of the sensor 70 and the cultivation level.

When the cultivation proceeds normally, cultures (pseudomonas) gradually change color from colorlessness to pink and then from pink to purple. As cultures are cultured, the color gradually changes, and at a certain color (color range), the cultivation should be stopped. As time passes and the cultivation proceeds, the color of the culture solution is changed, and there is a problem in that since the cultivation time and cultivation level are not always proportional, the color of the cultures should be continuously obtained, and the cultivation should be stopped at a certain color.

In the data set for pre-training, the ‘cultivation level’ may be divided into a plurality of cultivation levels.

For example, the cultivation level may be divided from a first level to a sixth level, and among them, a certain level (for example, a fourth level) may be an ‘appropriate level’. The sensing data may be the color image of the sensor 70 capturing the culture solution. The data pair of ‘color image of the culture solution-certain cultivation level’ may be used as learning data when the machine learning model is pre-trained. A large number of data pairs are secured as the data set, and then the machine learning model is trained.

The control unit receives the sensing data from the sensor 70 and inputs the sensing data into the machine learning model pre-trained, and the machine learning model may output the cultivation level. When the machine learning model determines (output) that the cultivation level is the ‘appropriate level’, the control unit may stop the driving part or notify a user.

The cultivation may not proceed as a normal process, and when the color of the culture solution is red, the culture solution lacks oxygen. When the machine learning model is trained with the data set including the data pair consisting of the sensing data of the sensor 70 and the cultivation level, the color image of the red-colored culture solution and the cultivation level of ‘lack of oxygen’ match with each other and are included into the data set.

The present disclosure provides a bioreactor for mass production shaking multi culture plates at the same time. According to the embodiment of the present disclosure, among the movements of the multi-station bioreactor, the machine learning model of the control unit determines a lack of oxygen, the control unit may increase the operation speed of the driving part or notify the user so that the user takes an action. The operation speed of the driving part is increased, which increases shaking and the amount of dissolved oxygen.

According to the present disclosure, the multi-station bioreactor has the advantage of culturing a large number of microorganisms such as pseudomonas with the simple and low-cost structure.

Meanwhile, the single large-capacity bioreactor is a fixed type and should use a rotating place and the like. Therefore, there are disadvantages of large cell stress and damage increased cross-contamination possibility, and low process flexibility. On the other hand, the multi-station bioreactor of the present disclosure can perform large-capacity cultivation without the rotating blade, so the multi-station bioreactor has advantages such as no cell stress or damage, low cross-contamination possibility, and high process flexibility. Since the culture solution is separately placed for each culture container, contamination and problems in a certain culture container are not transferred to other culture containers.

Furthermore, according to the present disclosure, the multi-station bioreactor has a structure in which a new column may be coupled to the existing columns as illustrated in FIG. 6 to increase capacity, and there is the advantage of having the space efficiency and facilitating up scaling.

What is claimed is:

1. A multi-station bioreactor comprising:
a plurality of stations (10) on which a culture container (1) containing culture solution is placed; and
a driving part (30) generating a mechanical driving force,
wherein the plurality of stations (10) is arranged to be vertically spaced apart from each other, and
the driving force of the driving part (30) is transmitted to the plurality of stations (10) so that the plurality of stations (10) is moved at the same time.

2. The multi-station bioreactor of claim 1, wherein each of the plurality of stations (10) is rotatably hinged with a standing frame (50) at facing two left and right hinge points of each station (10),
the multi-station bioreactor further comprises: a connecting rod (20) connected at one end to the driving part (30), and connected to a connection point of each of the plurality of stations (10), the connection point being spaced apart from each of the two hinge points, and transmitting the driving force of the driving part (30) to each of the stations (10).

3. The multi-station bioreactor of claim 2, wherein each of the stations (10) is moved like a seesaw as the connection point is rotated at a predetermined angle on each of the two hinge points by the connecting rod (20).

4. The multi-station bioreactor of claim 3, wherein the driving part (30) comprises:
a motor (31) driven by electric energy;
a reducer (32) reducing and outputting rotation of the motor; and
a rotating wheel (33) coupled to an output terminal of the reducer (32),
wherein one end of the connecting rod (20) is connected to circumference of the rotating wheel (33) so that the end of the connecting rod (20) is circularly moved.

5. The multi-station bioreactor of claim 2, wherein the driving part (30) comprises a vibrator generating vibration, and
one end of the connecting rod (20) is coupled to the vibrator, and the connecting rod (20) transmits the vibration to the connection point.

6. The multi-station bioreactor of claim 1, further comprising:
a heating part (81) provided on an upper surface of each station (10) or a bottom of the culture container (1) and consisting of a heating wire or a heating plate to heat the culture solution.

7. The multi-station bioreactor of claim 1, wherein pseudomonas is cultured.

8. The multi-station bioreactor of claim 1, further comprising:
a sensor (70) sensing at least color of the culture solution in the culture container (1); and
a control unit comprising a machine learning model that determines a cultivation level from sensing data of the sensor (70).

9. The multi-station bioreactor of claim 8, wherein the machine learning model is pre-trained with a data set including a data pair consisting of the sensing data of the sensor (70) and the cultivation level.

10. The multi-station bioreactor of claim 9, wherein the cultivation level includes an appropriate level, and
when the machine learning model determines the cultivation level as the appropriate level, the machine learning model stops the driving part or notifies a user.

11. The multi-station bioreactor of claim 9, wherein the cultivation level includes a lack of oxygen, and
when the machine learning model determines the cultivation level as the lack of oxygen, the control unit increases operation speed of the driving part or notifies the user.
